(19) United States
(12) Patent Application Publication
(10) Pub. No.: US 2022/0071546 A1
(43) Pub. Date: Mar. 10, 2022

(54) ELECTROCARDIOGRAM SIGNAL PROCESSING APPARATUS, METHOD, AND PROGRAM FOR IDENTIFYING SUPRAVENTRICULAR ARRHYTHMIA AND VENTRICULAR ARRHYTHMIA

(71) Applicant: ATSENS CO., LTD., Gyeonggi-do (KR)

(72) Inventors: Kab Mun CHA, Seoul (KR); Jong Ook JEONG, Gyeonggi-do (KR)

(73) Assignee: ATSENS CO., LTD., Gyeonggi-do (KR)

(21) Appl. No.: 17/462,720

(22) Filed: Aug. 31, 2021

(30) Foreign Application Priority Data
Sep. 4, 2020 (KR) 10-2020-0113200

(51) Int. Cl. A61B 5/364; A61B 5/352; A61B 5/024

(52) U.S. Cl. CPC A61B 5/364; A61B 5/024; A61B 5/352

(57) ABSTRACT

Provided are an electrocardiogram signal processing apparatus and method for identifying supraventricular arrhythmia and ventricular arrhythmia particularly to determine, by using the morphological similarity, complexity value, and R-R interval length of an electrocardiogram signal of a subject, regardless of whether or not the subject has supraventricular arrhythmia or ventricular arrhythmia.

### CROSS-REFERENCE TO RELATED APPLICATION

This application is based on and claims priority under 35 U.S.C. § 119 to Korean Patent Application No. 10-2020-0113200, filed on Sep. 4, 2020, in the Korean Intellectual Property Office, the disclosure of which is incorporated by reference herein in its entirety.

### BACKGROUND

### 1. Field

One or more embodiments relate to an electrocardiogram signal processing apparatus and a method for identifying supraventricular arrhythmia and ventricular arrhythmia, and more particularly, to a technique for identifying supraventricular arrhythmia and ventricular arrhythmia of a subject by using morphological similarity, complexity value, and R-R interval length of the electrocardiogram signal of the subject.

### 2. Description of the Related Art

Stand-alone or fixed electrocardiogram measuring devices of the related art are used to measure the electrocardiogram signal of a user in a state in which a user maintains a fixed posture and has a large gap (distance) between electrodes attached to the user. Thus, a baseline of the electrocardiogram signal is stable without large variations, and a magnitude of the electrocardiogram signal is large.

However, wearable patch-type electrocardiogram measuring devices have a small distance between electrodes and are subject to user's movements. Thus, in addition to human body noises, various types of noises caused by user's movements are included in electrocardiogram signals measured with such wearable patch-type electrocardiogram measuring devices. Furthermore, the baselines of such electrocardiogram signals may not be stable but vary greatly.

### SUMMARY

One or more embodiments include an electrocardiogram signal processing apparatus, method, and computer program for identifying supraventricular arrhythmia and ventricular arrhythmia of a user by using the morphological similarity, complexity value, and R-R interval length of the electrocardiogram signal of the subject.

Additional aspects will be set forth in part in the description which follows and, in part, will be apparent from the description, or may be learned by practice of the presented embodiments of the disclosure.

According to one or more embodiments, a method of identifying supraventricular arrhythmia and ventricular arrhythmia may include: sensing, by an electrocardiogram signal processing apparatus, an electrocardiogram signal of a subject; loading, by the electrocardiogram signal processing apparatus, a first signal segment of the electrocardiogram signal; calculating, by the electrocardiogram signal processing apparatus, morphological similarity to a reference template signal by comparing the first signal segment with the reference template signal; calculating, by the electrocardiogram signal processing apparatus, a Shannon entropy value of the first signal segment; and determining, by the electrocardiogram signal processing apparatus, whether the first signal segment is at least one selected from the group consisting of supraventricular arrhythmia and ventricular arrhythmia, the determining being performed by considering at least one selected from the group consisting of the morphological similarity, the Shannon entropy value, and an R-R interval length of the first signal segment.

In at least one variant, the electrocardiogram signal may be divided into signal segments by a multiple of a heart rate measurement time, and a signal segment having a highly frequent form among the signal segments may be the reference template signal.

In another variant, in the determining, the first signal segment may be determined as supraventricular arrhythmia when the morphological similarity is greater than a preset reference similarity value, the R-R interval length of the first signal segment is less than a corresponding dominant interval length, and the Shannon entropy value is greater than a first reference entropy value.

In the determining, the first signal segment may be determined as ventricular arrhythmia when the morphological similarity is less than a preset reference similarity value and the Shannon entropy value is greater than a second reference entropy value.

In further another variant, the reference template signal may be determined, based on frequencies of occurrence of signal segments among electrocardiogram signals of the subject, from among electrocardiogram signals having a frequency equal to or greater than a preset maximum frequency value.

In another variant, the dominant interval length may be determined based on one of preset interval length values, one of average values of R-R interval length values around the first signal segment, and one of average values of interval length values of all signal segments of the electrocardiogram signal.

In another variant, the dominant interval length may be determined based on a trend of R-R interval length values of signals of the electrocardiogram signal, the signals corresponding to the reference template signal.

In another variant, the dominant interval length may be a length obtained by applying an interpolation method to R-R interval length values of the electrocardiogram signal.

In another variant, when the electrocardiogram signal processing apparatus determines the first signal segment as supraventricular arrhythmia or ventricular arrhythmia, the method may further include generating and inserting, by the electrocardiogram signal processing apparatus, an “arrhythmia” tag into the first signal segment.

According to one or more embodiments, a computer program may be stored in a non-transitory computer-readable storage medium for executing the method by using a computer.

In addition, other methods and other systems for implementing the present disclosure, and non-transitory computer-readable recording media having recorded thereon computer programs for executing the other methods may be provided.

Other aspects, features, and advantages will become apparent and more readily appreciated from the accompanying drawings, claims, and detailed description.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram illustrating an electrocardiogram signal processing apparatus according to an embodiment;

FIG. 2 is a flowchart illustrating electrocardiogram signal processing methods according to embodiments;

FIG. 3 is a flowchart of a method for determining whether a supraventricular arrhythmia is present according to embodiments of the present disclosure;

FIG. 4 is a flowchart of a method for determining whether a ventricular arrhythmia is present according to embodiments of the present disclosure;

FIG. 5 is a flowchart illustrating a method of determining a reference template signal;

FIG. 6 is a diagram illustrating a process of determining a dominant interval length;

FIG. 7 is a graph illustrating a heart rate variation trend of an electrocardiogram signal; and

FIG. 8 is a diagram illustrating view data transmission between electrocardiogram signal processing apparatus, electrocardiogram sensing apparatus, and electronic apparatus.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, configurations and operations of the present disclosure will be described according to embodiments with reference to the accompanying drawings.

The present disclosure may be variously modified and may have various embodiments, and some embodiments illustrated in the accompanying drawings will now be described. Effects and features of the present disclosure, and implementation methods thereof will be clarified through the following embodiments described with reference to the accompanying drawings. However, the scope and idea of the present disclosure are not limited to the following embodiments but may be implemented in various forms.

Hereinafter, embodiments of the present disclosure will be described with reference to the accompanying drawings. In the following description given with reference to the accompanying drawings, the same elements or corresponding elements are denoted with the same reference numerals, and overlapping descriptions thereof will be omitted.

In the present specification, terms such as “training” and “learning” are not used to refer to mental actions such as educational activities of humans, but are used to refer machine learning through computing procedures.

In the following embodiments, terms such as first and second are not used in a limiting sense, but are used for the purpose of distinguishing one element from other elements.

In the following embodiments, the terms of a singular form may include plural forms unless referred to the contrary.

In addition, terms such as “include” or “comprise” specify features or the presence of stated elements, but do not exclude one or more other features or elements.

In the drawings, the sizes of elements may be exaggerated or reduced for ease of description. For example, in the drawings, the size or thickness of each element may be arbitrarily shown for illustrative purposes, and thus the present disclosure should not be construed as being limited thereto.

The order of processes explained in one embodiment may be changed in a modification of the embodiment or another embodiment. For example, two consecutively described processes may be performed substantially at the same time or performed in an order opposite to the described order.

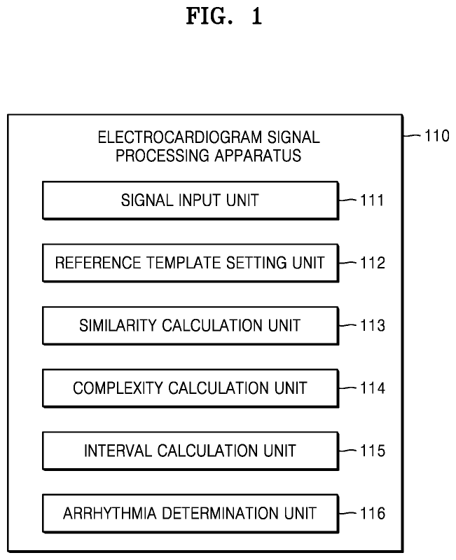

FIG. 1 is a block diagram illustrating an electrocardiogram signal processing apparatus 110 according to an embodiment.

The electrocardiogram signal processing apparatus 110 may include a signal input unit 111, a reference template setting unit 112, a similarity calculation unit 113, a complexity calculation unit 114, an interval calculation unit 115, and an arrhythmia determination unit 116.

The signal input unit 111 receives an electrocardiogram signal. The signal input unit 111 may receive an electrocardiogram signal from an external electronic device. The electrocardiogram signal may be measured and received by an external device or may be measured and received by a measuring unit provided therein, but is not limited thereto. The signal input unit 111 may divide the electrocardiogram signal into signal segments. The electrocardiogram signal may be divided into signal segments based on the form of QRS.

The reference template setting unit 112 may determine a reference template signal for the electrocardiogram signal. The reference template setting unit 112 may determine a reference template signal using signal segments. The reference template setting unit 112 may convert the signal segments into morphological patterns, classify the signal segments according to the morphological patterns, and determine the reference template signal using the classification result. For example, the reference template setting unit 112 may set all or part of a signal segment including the most frequent morphological pattern as the reference template signal.

The reference template setting unit 112 may divide the electrocardiogram signal into as many signal segments as a multiple of a heart rate measurement time, and set the most frequent signal segment among the signal segments as the reference template signal. For example, when a heart rate is 60 beats/min and the measurement time period of the electrocardiogram signal is 1 hour, the electrocardiogram signal may be divided into 3600 (60 minutes×60 times) signal segments. The 3600 signal segments may be classified according to the morphological patterns thereof, and the most frequent morphological may be determined as the reference template signal. The reference template signal is a signal determined from electrocardiogram signals measured for each object, and may be determined based on a representative signal segment of each electrocardiogram signal. The representative signal segment may be determined based on the frequency of occurrence, but is not limited thereto, and may be determined in consideration of the occurrence probability, the pattern in the electrocardiogram signal, and the occurrence probability of the pattern.

The similarity calculation unit 113 calculates the morphological similarity between the reference template signal and a first signal segment of the electrocardiogram signal. When the Pearson correlation coefficient between values of the first signal segment and values of the reference template signal is equal to or greater than a preset reference coefficient value, the similarity calculation unit 113 determines that the first signal segment is similar to the reference template signal.

The complexity calculation unit 114 calculates a complexity value, such as a Shannon entropy value, based on the values of the first signal segment.

Here, the complexity value may be calculated using a complexity calculation algorithm such as Shannon entropy. However, calculation of the complexity value is not limited thereto and may be performed by various other methods such as Kolmogorov complexity, monotone complexity, prefix complexity, and decision complexity.

The complexity value may be determined based on the frequencies of occurrence of magnitude ranges of measured values of the data of the electrocardiogram signal. The complexity value may be determined based on the frequency of occurrence of a specific measured value of the electrocardiogram signal or the first signal segment. The measured values of the electrocardiogram signal may be grouped into a plurality of magnitude bins based on the magnitude of the measured values. For example, the measured values of the electrocardiogram signal may be grouped into a first magnitude bin, a second magnitude bin, a third magnitude bin, . . . , and an nth magnitude bin based on the maximum and minimum of the measured values. Each magnitude bin may be defined as a set of one or more values or may be defined as a single value. In this case, a first frequency of occurrence may be obtained by extracting data (points) of the electrocardiogram signal which is included in the first magnitude bin. The case in which a single magnitude bin has a high frequency of occurrence may mean that the electrocardiogram signal (data) is generated in similar patterns, and the case in which each magnitude bin has a low frequency of occurrence may mean that the electrocardiogram signal has irregular patterns, that is, a complex form.

In other words, the complexity value may be calculated using Equation 1 below by defining at least one magnitude bin based on the measured values of the electrocardiogram signal, and calculating the frequency of occurrence of data points in each magnitude bin.

$$SE = - {\sum }_{m = 1}^{M} p ( m ) {log}_{2} p ( m ) \qquad [ Equation 1 ]$$

Here, M refers to the number of magnitude bins of a signal, and p(m) refers to a probability function of occurrence of the electrocardiogram signal in each magnitude bin. For example, when m is 1, p(m) may refer to the ratio of a data point number N corresponding to m=1 to a total data point number L.

$$p ( m ) = \frac{N}{L}$$

For example, m may refer to a measured value defined by 8 bits, and in this case in which the number of bits defining each measured value is 8, M may be 255 (=Cambria Math) which is the maximum of measured values. L may refer to the number of signal segments included in the electrocardiogram signal. For example, L may refer to the total number of data points, and when sampling is performed at a preset sampling frequency for the total time length of the electrocardiogram signal, L may be calculated by dividing the time length by the sampling frequency. N may refer to the number of occurrences of a specific value in the segments of the electrocardiogram signal.

The interval calculation unit 115 determines the R-R interval length of the first signal segment of the electrocardiogram signal. The R-R interval length may be determined based on the QRS form of the first signal segment. The interval calculation unit 115 may output whether the R-R interval length of the first signal segment is equal to or less than a preset dominant interval length.

The dominant interval length may be determined based on one of preset interval length values, one of the average values of R-R interval length values around the first signal segment, and one of the average values of interval length values of all the signal segments of the electrocardiogram signal. The dominant interval length may be determined as a length value obtained by applying interpolation to R-R interval length values of the electrocardiogram signal. The expression “around the first signal segment” refers to one or more segments adjacent to the first signal segment. The average value of interval lengths of as many signal segments as a manager has set may be determined as the dominant interval length. In addition, the dominant interval length may be determined based on present fractions of one of preset interval length values, one of the average values of R-R interval length values around the first signal segment, and one of the average values of interval length values of all the signal segments of the electrocardiogram signal. The preset fractions may be, for example, about 50%, about 90%, and 120%, but are not limited thereto.

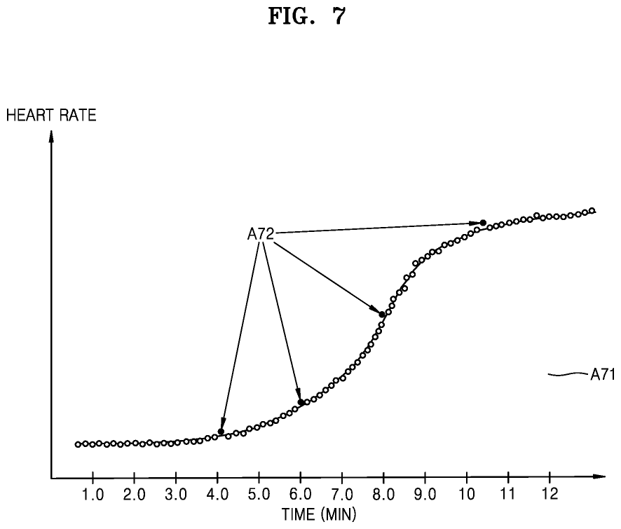

In a trend of heart rate variations caused by situations of a subject as shown in FIG. 7, the interval calculation unit 115 may determine whether the R-R interval length of a given signal segment is equal to or less than the preset dominant interval length. Because a heart rate and an R-R interval length have an inverse relationship, a signal segment having an R-R interval length equal to or less than the dominant interval length may be determined based on a heart rate. FIG. 7 is a graph showing a trend A71 of heart rate variations with respect to time. Because the trend A71 of heart rate variations depends on the heart condition of the subject, different subjects may have different trends of heart rate variations. A signal segment of which the R-R interval length is equal to less than the dominant interval length may be determined as A72 by comparison with the trend A71 of heart rate variations. In this case, the dominant interval length may be determined according to the heart rate of the signal segment A72. To indicate that the R-R interval length of the signal segment A72 is equal to or less than the dominant interval length, the interval calculation unit 115 may output “TRUE” for the signal segment A72.

The arrhythmia determination unit 116 may determine whether the first signal segment corresponds to supraventricular arrhythmia or ventricular arrhythmia.

Based on the morphological similarity of the first signal segment, the complexity value of the electrocardiogram signal, and the R-R interval length of the first signal segment, the arrhythmia determination unit 116 may determine whether the first signal segment corresponds to supraventricular arrhythmia.

When the morphological similarity of the first signal segment is equal to or less than a preset first reference similarity value, the R-R interval length of the first signal segment is equal to or less than the dominant interval length, and the complexity value of the first signal segment is equal to or greater than a first reference complexity value, the arrhythmia determination unit 116 may determine the heart condition of the subject as supraventricular arrhythmia. The first signal segment may be tagged as supraventricular arrhythmia (SVA) according to the determination of the arrhythmia determination unit 116.

Based on the morphological similarity of the first signal segment and the Shannon entropy value of the electrocardiogram signal, the arrhythmia determination unit 116 may determine whether the heart condition of the subject corresponds to ventricular arrhythmia. When the morphological similarity of the first signal segment is equal to or less than a second reference similarity value, and the complexity value of the first signal segment is equal to or greater than a second reference complexity value, the arrhythmia determination unit 116 may determine the heart condition of the subject as ventricular arrhythmia (VA). The first signal segment may be tagged as ventricular arrhythmia (VA).

As described above, the electrocardiogram signal processing apparatus 110 of the embodiment may analyze the electrocardiogram signal of a subject and may determine whether the heart condition of the subject corresponds to supraventricular arrhythmia or ventricular arrhythmia.

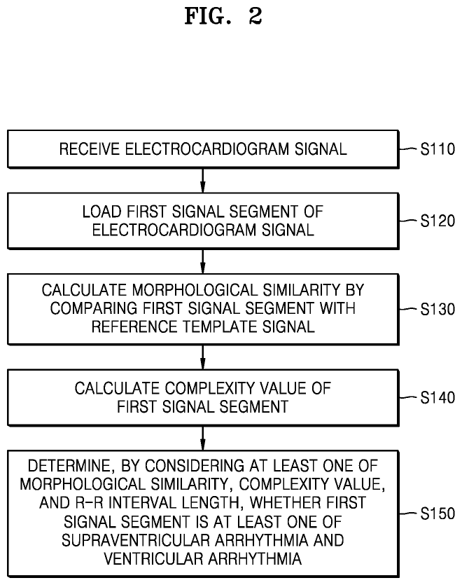

FIG. 2 is a flowchart illustrating electrocardiogram signal processing method of determining supraventricular arrhythmia or ventricular arrhythmia by calculating the similarity and complexity of electrocardiogram signal according to embodiments.

In operation S110, the electrocardiogram signal processing apparatus 110 receives an electrocardiogram signal. The electrocardiogram signal processing apparatus 110 may receive an electrocardiogram signal from an external device. The electrocardiogram signal processing apparatus 110 may receive an electrocardiogram signal measured by a measuring unit provided therein.

In operation S120, the electrocardiogram signal processing apparatus 110 divides the electrocardiogram signal into signal segments and loads a first signal segment from the signal segments. The electrocardiogram signal may be divided into signal segments based on the form of QRS.

In operation S130, the electrocardiogram signal processing apparatus 110 compares the first signal segment with a reference template signal to calculate morphological similarity. The electrocardiogram signal processing apparatus 110 calculates the morphological similarity between the first signal segment of the electrocardiogram signal and the reference template signal. When the Pearson correlation coefficient between values of the first signal segment and values of the reference template signal is equal to or greater than a preset reference coefficient value, the electrocardiogram signal processing apparatus 110 determines that the first signal segment is similar to the reference template signal.

In operation S140, the electrocardiogram signal processing apparatus 110 calculates a complexity value of the first signal segment. The electrocardiogram signal processing apparatus 110 calculates a complexity value such as a Shannon entropy value based on the values of the first signal segment.

Here, the complexity value may be calculated using a complexity calculation algorithm such as Shannon entropy. However, the complexity value is not limited thereto and may be calculated by various methods such as Kolmogorov complexity, monotone complexity, prefix complexity, and decision complexity. The process of calculating the complexity value is the same as the operation of the complexity calculation unit 114, and thus a description thereof will not be presented here.

By considering at least one of the morphological similarity of the first signal segment, the complexity value of the first signal segment, and the R-R interval length of the first signal segment, the electrocardiogram signal processing apparatus 110 determines whether the first signal segment is at least one of supraventricular arrhythmia and ventricular arrhythmia. The electrocardiogram signal processing apparatus 110 generates a tag for the first signal segment in consideration of at least one of a morphological similarity to the first signal segment, a complexity value of the first signal segment, and an RR interval length of the first signal segment. The electrocardiogram signal processing apparatus 110 may insert the generated tag to the electrocardiogram signal or the first signal segment.

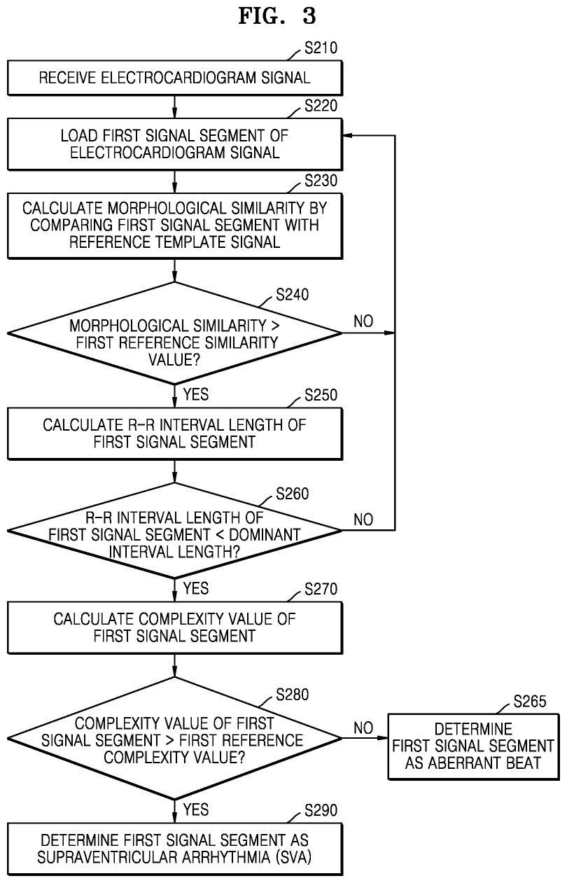

FIG. 3 is a flowchart of a method for determining whether a supraventricular arrhythmia is present according to embodiments of the present disclosure.

In operation S210, the electrocardiogram signal processing apparatus 110 receives an electrocardiogram signal.

In operation S220, the electrocardiogram signal processing apparatus 110 loads a first signal segment of the electrocardiogram signal.

In operation S230, the electrocardiogram signal processing apparatus 110 compares the first signal segment with a reference template signal to calculate morphological similarity.

In operations S240 and S250, when the morphological similarity of the first signal segment is greater than a preset first reference similarity value, the electrocardiogram signal processing apparatus 110 calculates the R-R interval length of the first signal segment. The electrocardiogram signal processing apparatus 110 determines the R-R interval length of the first signal segment of the electrocardiogram signal. The R-R interval length may be determined based on the QRS form of the first signal segment. The electrocardiogram signal processing apparatus 110 may output whether the R-R interval length of the first signal segment is equal to or less than a preset dominant interval length.

In a trend of heart rate variations caused by movements of a subject as shown in FIG. 7, the electrocardiogram signal processing apparatus 110 may determine whether the R-R interval length of a given signal segment is equal to or less than the preset dominant interval length. A heart rate and an R-R interval length have an inverse relationship. FIG. 7 is a graph showing a trend A71 of heart rate variations with respect to time. Because the trend A71 of heart rate variations depends on the heart condition of the subject, different subjects may have different trends of heart rate variations. A signal segment of which the R-R interval length is equal to or less than the dominant interval length may be determined as A72 by comparison with the trend A71 of heart rate variations. In this case, the dominant interval length may be determined according to the heart rate of the signal segment A72. To indicate that the R-R interval length of the signal segment A72 is equal to or less than the dominant interval length, the interval calculation unit 115 may output “TRUE” for the signal segment A72.

In operation S260, the electrocardiogram signal processing apparatus 110 determines whether the R-R interval length of the first signal segment is less than the dominant interval length.

In operation S270, the electrocardiogram signal processing apparatus 110 calculates a complexity value of the first signal segment.

Here, the complexity value may be calculated using a complexity calculation algorithm such as Shannon entropy. However, the complexity value is not limited thereto and may be calculated by various methods such as Kolmogorov complexity, monotone complexity, prefix complexity, and decision complexity. The process of calculating the complexity value is the same as the operation of the complexity calculation unit 114, and thus a description thereof will not be presented here.

In operation S280, the electrocardiogram signal processing apparatus 110 determines whether the complexity value of the first signal segment is greater than a preset first reference complexity value.

In operation S290, when the complexity value of the first signal segment is greater than the preset first reference complexity value, the electrocardiogram signal processing apparatus 110 determines that the first signal segment is supraventricular arrhythmia (SVA). The electrocardiogram signal processing apparatus 110 may add a tag of supraventricular arrhythmias to the first signal segment. The tag may be added to correspond to the first signal segment or may be added to the electrocardiogram signal by including information on the first signal segment.

When the complexity value of the first signal segment is equal to or less than the preset first reference complexity value, the first signal segment may be determined as an aberrant beat.

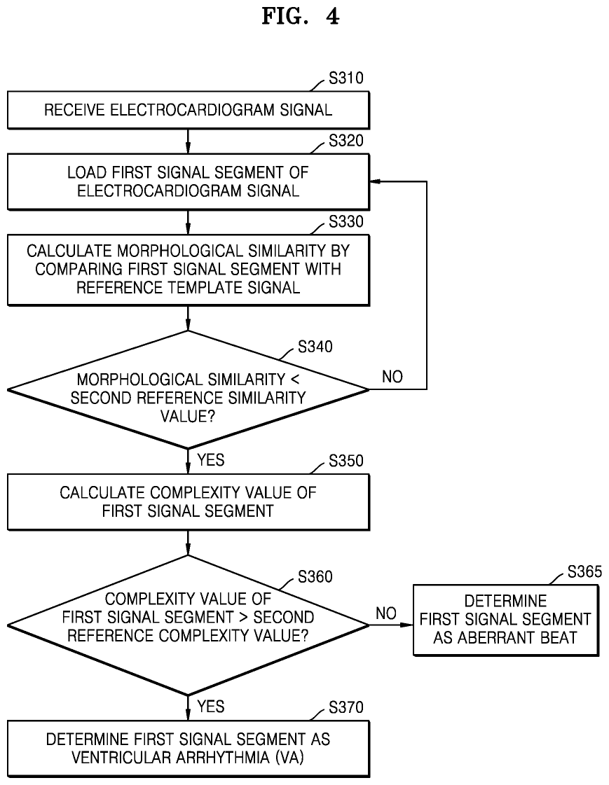

FIG. 4 is a flowchart of a method for determining whether a ventricular arrhythmia is present according to embodiments of the present disclosure.

In operation S310, the electrocardiogram signal processing apparatus 110 receives an electrocardiogram signal.

In operation S320, the electrocardiogram signal processing apparatus 110 loads a first signal segment of the electrocardiogram signal.

In operation S330, the electrocardiogram signal processing apparatus 110 compares the first signal segment with a reference template signal to calculate morphological similarity.

In operation S340, the electrocardiogram signal processing apparatus 110 determines whether the morphological similarity of the first signal segment is less than a preset second reference similarity value.

In operation S350, when the morphological similarity to the first signal segment is less than the preset second reference similarity value, the electrocardiogram signal processing apparatus 110 calculates a complexity value of the first signal segment. Because the process of calculating the complexity value is the same as the operation of the complexity calculation unit 114, a description thereof will not be presented here.

In operation S360, the electrocardiogram signal processing apparatus 110 determines whether the complexity value of the first signal segment is greater than a preset second reference complexity value.

In operation S370, the electrocardiogram signal processing apparatus 110 determines that the heart condition of a subject is ventricular arrhythmia (VA). The electrocardiogram signal processing apparatus 110 may add a ventricular arrhythmia tag to the first signal segment. The tag may be added to correspond to the first signal segment or may be added to the electrocardiogram signal by including information on the first signal segment.

When the complexity value of the first signal segment is equal to or less than the preset second reference complexity value, the second signal segment may be determined as an aberrant beat.

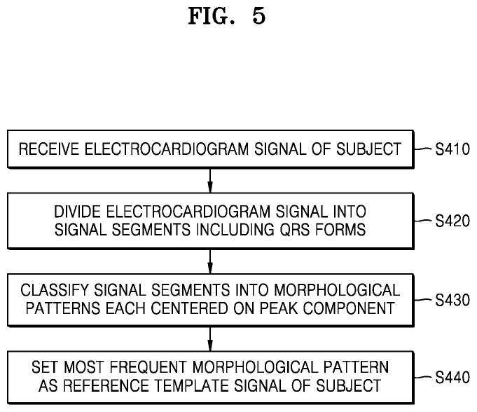

FIG. 5 is a flowchart illustrating a method of determining a reference template signal.

In operation S410, the electrocardiogram signal processing apparatus 110 receives an electrocardiogram signal of a subject.

In operation S420, the electrocardiogram signal processing apparatus 110 divides the electrocardiogram signal into signal segments each including a QRS form.

In operation S430, the electrocardiogram signal processing apparatus 110 classifies the signal segments into morphological patterns each centered on a peak component.

In operation S440, the electrocardiogram signal processing apparatus 110 may determine a reference template signal by selecting one of the morphological patterns based on the frequency of occurrence. The most frequent morphological pattern among the morphological patterns of the electrocardiogram signal may be set as the reference template signal for the subject.

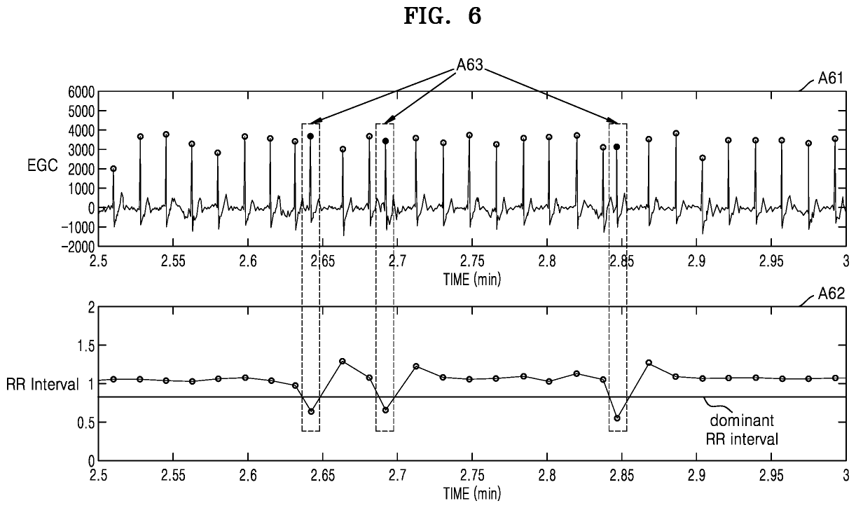

FIG. 6 is a diagram illustrating a process of determining a dominant interval length.

The electrocardiogram signal processing apparatus 110 may receive an electrocardiogram signal and divide the electrocardiogram signal into signal segments based on peak components of the electrocardiogram signal (refer to A61). The signal segments may each include an R peak. The electrocardiogram signal processing apparatus 110 may calculate a length between the R peaks based on the times when the R peaks are generated.

The electrocardiogram signal processing apparatus 110 may calculate R-R interval lengths based on the R peak of A61, respectively. The electrocardiogram signal processing apparatus 110 may convert the R-R interval lengths of the signal segments of the electrocardiogram signal into an interval length-time graph (refer to A62).

The interval calculation unit 115 of the electrocardiogram signal processing apparatus 110 may extract signal segments A63 having R-R interval lengths equal to or less than a dominant interval length (dominant R-R interval).

FIG. 7 is a graph illustrating a heart rate variation trend of an electrocardiogram signal.

The electrocardiogram signal processing apparatus 110 may generate a heart rate variation trend A71 by arranging the heart rate of the electrocardiogram signal with respect to time.

The electrocardiogram signal processing apparatus 110 may extract data (A72) having heart rates greater than the heart rate of the heart rate change trend A71. The interval calculation unit 115 of the electrocardiogram signal processing apparatus 110 may determine signal segments A72 based on the heart rates of signal segments such that signal segments having heart rates greater than a preset heart rate may be determined as signal segments A72.

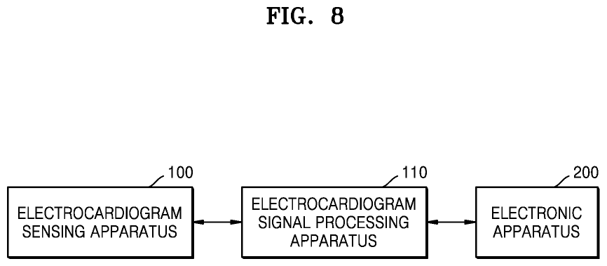

FIG. 8 is a diagram illustrating view data transmission between the electrocardiogram signal processing apparatus 110, the electrocardiogram sensing apparatus 100, and the electronic apparatus 200.

The electrocardiogram signal processing apparatus 110 may be electrically connected to the electrocardiogram sensing apparatus 100 or may be connected to the electrocardiogram sensing apparatus 100 through a network. The electrocardiogram signal processing apparatus 110 may receive an electrocardiogram signal from the electrocardiogram sensing apparatus 100.

The electrocardiogram signal processing apparatus 110 may be connected to the electrocardiogram sensing apparatus 100 and the electronic apparatus 200 by an electrical method or through a network. The electrocardiogram signal processing apparatus 110 may receive an electrocardiogram signal from the electrocardiogram sensing apparatus 100, and may transmit results of determination of supraventricular arrhythmia or ventricular arrhythmia to the electronic apparatus 200.

The electrocardiogram signal processing apparatus 110 may transmit, to the electronic apparatus 200, the received electrocardiogram signal and/or results of determination of whether the electrocardiogram signal corresponds to arrhythmia.

The electrocardiogram signal processing apparatus 110 may be included in the electrocardiogram sensing apparatus 100. The electrocardiogram signal processing apparatus 110 may generate data about results of determination of supraventricular arrhythmia or ventricular arrhythmia. The electrocardiogram signal processing apparatus 110 may record data including results of determination of supraventricular arrhythmia or ventricular arrhythmia and may transmit the data to the circuit unit 200 when the circuit unit 200 requests the data.

The above-described apparatuses or devices may be implemented with hardware elements, software elements, and/or combinations of hardware and software elements. For example, apparatuses, devices, methods, and elements described in the above embodiments may be implemented with at least one general-purpose or special-purpose computer such as a processor, a controller, an arithmetic logic unit (ALU), a digital signal processor, a microcomputer, a field programmable gate array (FPGA), a programmable logic unit (PLU), a microprocessor, or any other device capable of performing instructions and responding to instructions. A processing device (apparatus) may execute an operating system (OS) and at least one software application running on the operating system. In addition, the processing device may access, store, manipulate, process, and generate data in response to execution of software. For ease of understanding, the case of using a single processing device may be described. However, those of ordinary skill in the art will recognize that the processing device may include a plurality of processing elements and/or a plurality of types of processing elements. For example, the processing device may include a plurality of processors, or a processor and a controller. Other processing configurations such as parallel processors may also be possible.

Software may include a computer program, a code, an instruction, or a combination of at least one thereof. In addition, processing devices may be configured to operate in a desired manner and may be independently or collectively instructed. Software and/or data may be permanently or temporarily embodied in a certain machine, a component, a physical device, virtual equipment, a computer storage medium or device, or propagating signal waves so as to be interpreted by a processing device or provide instructions or data to the processing device. Software may be distributed over network coupled computer systems and may be stored and executed in a distributed fashion. Software and data may be stored in at least one non-transitory computer-readable recording medium.

The methods of the embodiments may be implemented in the form of program instructions executable on various computers and may then be stored in non-transitory computer-readable recording media. The non-transitory computer-readable recording media may include, individually or in combination, program instructions, data files, data structures, etc. The program instructions stored in the media may be those designed and configured according to the embodiments or well known in the computer software industry. The non-transitory computer-readable recording media include hardware specifically configured to store program instructions and execute the program instructions, and examples of the hardware include magnetic media such as hard disks, floppy disks, and magnetic tapes; optical media such as CD-ROMs and DVDs; magneto-optical media such as floptical disks; and ROMs, RAMs, and flash memories. Examples of the program instructions may include machine codes made by compilers and high-level language codes executable on computers using interpreters. The above-mentioned hardware device may be configured to operate via one or more software modules to perform operations according to embodiments, and vice versa.

According to the embodiments of the present disclosure, whether a subject has supraventricular arrhythmia or ventricular arrhythmia may be determined by using the morphological similarity, complexity value, and R-R interval length of an electrocardiogram signal. In particular, the embodiments of the present disclosure may be useful to identifying ventricular tachycardia.

Although some embodiments have been described with reference to the accompanying drawings, those skilled in the art various will understand that various modifications and changes are possible from the embodiments. For example, the above-described techniques may be performed in orders different from the described orders, and/or elements of the described systems, structures, devices, circuits or the like may be coupled or combined with each other in manners different from the above-described manners or may be replaced with other elements or equivalents thereof. In these cases, however, intended results may be achieved.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of identifying supraventricular arrhythmia and ventricular arrhythmia, the method comprising:
sensing, by an electrocardiogram signal sensing apparatus, an electrocardiogram signal of a subject;
loading, by an electrocardiogram signal processing apparatus, a first signal segment of the electrocardiogram signal;
calculating, by the electrocardiogram signal processing apparatus, morphological similarity to a reference template signal by comparing the first signal segment with the reference template signal;
calculating, by the electrocardiogram signal processing apparatus, a Shannon entropy value of the first signal segment; and
determining, by the electrocardiogram signal processing apparatus, whether the first signal segment is indicative of supraventricular arrhythmia or ventricular arrhythmia based on morphological similarity, the Shannon entropy value, an R-R interval length of the first signal segment, or a combination thereof.

2. The method of claim 1, further comprising:
dividing the electrocardiogram signal into signal segments by a multiple of a heart rate measurement time;
wherein a signal segment having a highly frequent form among the signal segments includes the reference template signal.

3. The method of claim 1, wherein determining whether the first signal segment is indicative of supraventricular arrhythmia or ventricular arrhythmia further comprises determining the first signal segment as supraventricular arrhythmia when:
the morphological similarity is greater than a preset reference similarity value,
the R-R interval length of the first signal segment is less than a corresponding dominant interval length, and
the Shannon entropy value is greater than a first reference entropy value.

4. The method of claim 1, wherein whether the first signal segment is indicative of supraventricular arrhythmia or ventricular arrhythmia further comprises determining the first signal segment as ventricular arrhythmia when:
the morphological similarity is less than a preset reference similarity value, and
the Shannon entropy value is greater than a second reference entropy value.

5. The method of claim 1, wherein the reference template signal is determined, based on frequencies of occurrence of signal segments among electrocardiogram signals of the subject, from among electrocardiogram signals having a frequency equal to or greater than a preset maximum frequency value.

6. The method of claim 4, wherein the dominant interval length is determined based on one of preset interval length values, one of average values of R-R interval length values around the first signal segment, and one of average values of interval length values of all signal segments of the electrocardiogram signal.

7. The method of claim 4, wherein the dominant interval length is determined based on a trend of R-R interval length values of signals of the electrocardiogram signal, the signals corresponding to the reference template signal.

8. The method of claim 4, wherein the dominant interval length is a length obtained by applying an interpolation method to R-R interval length values of the electrocardiogram signal.

9. The method of claim 1, further comprising:
when the electrocardiogram signal processing apparatus determines the first signal segment as supraventricular arrhythmia or ventricular arrhythmia, generating and inserting, by the electrocardiogram signal processing apparatus, an arrhythmia labeled tag into the first signal segment.

10. A computer program stored in a non-transitory computer-readable storage medium for executing the method of claim 1 by using a computer.

11. An electrocardiogram signal processing apparatus for identifying supraventricular arrhythmia and ventricular arrhythmia by using an electrocardiogram signal, the electrocardiogram signal processing apparatus comprising:
a signal input unit configured to receive an electrocardiogram signal of a subject and load a first signal segment of the electrocardiogram signal;
a similarity calculation unit configured to calculate morphological similarity to a reference template signal by comparing the first signal segment with the reference template signal;
a complexity calculation unit configured to calculate a complexity value of the first signal segment; and
an arrhythmia determination unit configured to determine, based on the morphological similarity, the complexity value, an R-R interval length of the first signal segment, or a combination thereof, whether the first signal segment is indicative of supraventricular arrhythmia or ventricular arrhythmia.

12. The electrocardiogram signal processing apparatus of claim 11, wherein the electrocardiogram signal is divided into signal segments by a multiple of a heart rate measurement time, and a signal segment having a highly frequent form among the signal segments is the reference template signal.

13. The electrocardiogram signal processing apparatus of claim 11, wherein the arrhythmia determination unit is configured to determine the first signal segment as supraventricular arrhythmia when:
the morphological similarity is greater than a preset reference similarity value,
the R-R interval length of the first signal segment is less than a corresponding dominant interval length, and
the complexity value is greater than a first reference complexity value.

14. The electrocardiogram signal processing apparatus of claim 11, wherein the arrhythmia determination unit is configured to determine the first signal segment as ventricular arrhythmia when:
the morphological similarity is less than a preset reference similarity value,
the R-R interval length of the first signal segment is less than a corresponding dominant interval length, and
the complexity value is greater than a second reference complexity value.

15. The electrocardiogram signal processing apparatus of claim 11, wherein the reference template signal is determined, based on frequencies of occurrence of signal segments among electrocardiogram signals of the subject, from among electrocardiogram signals having a frequency equal to or greater than a preset maximum frequency value.

16. The electrocardiogram signal processing apparatus of claim 14, wherein the dominant interval length is determined based on one of preset interval length values, one of average values of R-R interval length values around the first signal segment, and one of average values of interval length values of all signal segments of the electrocardiogram signal.

17. The electrocardiogram signal processing apparatus of claim 14, wherein the dominant interval length is determined based on a trend of R-R interval length values of signals of the electrocardiogram signal, the signals corresponding to the reference template signal.

18. The electrocardiogram signal processing apparatus of claim 14, wherein the dominant interval length is a length obtained by applying an interpolation method to R-R interval length values of the electrocardiogram signal.

19. The electrocardiogram signal processing apparatus of claim 11, wherein when the arrhythmia determination unit determines the first signal segment as supraventricular arrhythmia or ventricular arrhythmia, the arrhythmia determination unit generates and inserts an arrhythmia labeled tag into the first signal segment.
